(19) United States
(12) Patent Application Publication
(10) Pub. No.: US 2011/0172706 A1
(43) Pub. Date: Jul. 14, 2011

(54) TOOL DELIVERY SYSTEMS AND METHODS OF USE

(75) Inventors: Gary Kappel, Acton, MA (US); Barry Weitzner, Acton, MA (US); Paul Smith, Smithfield, RI (US)

(73) Assignee: Boston Scientific Scimed, Inc.

(21) Appl. No.: 12/898,092

(22) Filed: Oct. 5, 2010

Related U.S. Application Data
(60) Provisional application No. 61/294,745, filed on Jan. 13, 2010.

(51) Int. Cl. A61B 17/062

(52) U.S. Cl. 606/223

(57) ABSTRACT

Tool delivery systems and methods of use are disclosed herein. The delivery systems include a case for transporting a flexible tool to a procedure site and a guide tube. In one aspect, the flexible tool has a first size and shape greater than the diameter of a lumen within the guide tube. Placing the flexible tool into the case changes the flexible tool into a second, smaller size and shape and allows delivery of the needle through the guide tube.

### CROSS REFERENCE TO RELATED APPLICATIONS

This application claims priority to U.S. Provisional Application No. 61/294,745, filed in the U.S. Patent and Trademark Office on Jan. 13, 2010, and incorporated herein by reference.

### BACKGROUND OF THE INVENTION

Conventional surgical tools, such as endoscopic and laparoscopic devices, can provide surgical access to localized procedure sites while minimizing patient trauma. Although the growing capabilities of such therapeutic devices allow users to perform an increasing variety of surgeries through traditional minimally invasive routes, further refinements may allow surgical access through even less invasive routes and/or facilitate conventional surgical procedures.

Constraints on the size of anatomic passageways, surgical incisions, and/or instrument channels create particular challenges. For example, the delivery of surgical instruments may be difficult or impossible. Accordingly, there is room for further refinement of tool delivery devices and methods.

### SUMMARY

As the sophistication of minimally invasive procedures develops, the desire to deliver surgical tools to a site within the human body has increased. In some cases the size and shape of surgical tools are reduced or altered to allow delivery through a channel of an access and visualization type system, trocar, guide tube, working channel or catheter. However, there are limits on the ability to scale medical devices without the loss of functionality. In addition, off the shelf tools may be preferred due to their availability, cost, and familiarity. One such example is the delivery of suture needles. The length, radius size, and/or radius arc height of many common suture needles prevent their delivery through the channel of a guide tube.

Disclosed herein are various devices for use in delivering a tool to a procedure site within a patient. In one exemplary embodiment the delivery device includes a case or housing with an inner cavity sized and shaped to receive at least a portion of a flexible tool. The housing can reduce the likelihood of the flexible tool snagging the walls of a guide tube (i.e., delivery tube) and allow maximum use of the guide tube channel width. In addition, the inner cavity of the device can constrain the flexible tool and hold the flexible tool in a low profile configuration. The device (e.g., case or container) can also protect both the tool being transported and the working channel the tool is being transport through. The case or container would allow for quick, safe, convenient and easy tool deployment. It may also allow the tool to be delivered in a sterile fashion without contamination by the access path. Further, it may also allow the tool to have a predicable orientation on exit.

In one aspect, the flexible tool has a curved body. The case can reduce the tool curvature such that the flexible tool has a smaller width compared to the flexible tool in the unconstrained configuration (i.e., outside of the case). In addition, the case can be sized and shaped for passage through a channel within another instrument. In one aspect, the case can fit within and travel through the lumen of a guide tube.

In one embodiment the flexible tool is a needle and the inner cavity of the case can include mating features for inhibiting relative movement between the needle and case. For example, a recess along the inner surface of the inner cavity can hold the needle in place while the case is transported to a surgical site. In another embodiment, a removable cover can cover a sharp end of the needle. The needle can disengage from the cover when removed from the case at the procedure site. The cover can be part of the case in one embodiment. Additionally, the terms cover, case, container, and capsule may be used interchangeably herein.

A method for delivering the needle can include inserting the needle into the case or providing a needle positioned within a case. While in the case, the needle is constrained such that the length, radius size, and/or radius arc height of the needle is reduced. The case is then delivered through a channel of a guide tube to a procedure site and removed from the case. In one aspect, the case is opened to remove the needle. Alternatively, the case includes an opening through which the needle can be removed. This delivery means is applicable to many different medical devices, for example: fasteners, cutting blades, injection needles, components for creating an internal bridgework or other type structures for tissue traction, supports, shielding, medical therapeutics, targeted drugs, and other devices to address permanent or temporary needs.

In one embodiment, once the needle is removed, it takes on a second shape that differs from the shape it comprised during transport. For example, the needle can assume an increased curvature, length, radius size, and/or radius arc height upon removal. The shape of the needle can accommodate such increases when the needle (i.e., flexible tool) comprises one or more curves, three-dimensional curves, coils, etc.

Additional objects and advantages of the embodiments will be set forth in part in the description which follows, and in part will be obvious from the description, or may be learned by practice of the embodiments. The objects and advantages of the embodiments will be realized and attained by means of the elements and combinations particularly pointed out in the appended claims.

It is to be understood that both the foregoing general description and the following detailed description are exemplary and explanatory only and are not restrictive of the embodiments, as claimed.

The accompanying drawings, which are incorporated in and constitute a part of this specification, illustrate several embodiments and together with the description, serve to explain the principles of the embodiments.

### BRIEF DESCRIPTION OF THE DRAWINGS

The accompanying drawings, which are incorporated in and constitute a part of this specification, illustrate several embodiments and together with the description, serve to explain the principles of the embodiments.

FIGS. 1A through 1E are partially transparent, perspective views of a delivery device described herein.

FIG. 2A is a front view of one exemplary embodiment of a device described herein;

FIG. 2B is a front view of another embodiment of the device of FIG. 2A.

FIG. 3A is a side view one exemplary embodiment of a flexible tool described herein;

FIG. 3B is a cross-sectional view of the flexible tool of FIG. 3A in a low profile configuration.

FIG. 4 is a perspective view of another exemplary embodiment of a delivery device disclosed herein.

FIG. 5 is a perspective view of yet another exemplary embodiment of a delivery device disclosed herein.

FIG. 6 is a partial perspective view of a system disclosed herein.

FIG. 7 is an exemplary flow chart comprising steps for delivering a flexible tool through an elongate device.

### DESCRIPTION OF THE EMBODIMENTS

Described below are exemplary embodiments of systems and devices for delivering a flexible tool to a target location, such as a surgical site. In one embodiment, a delivery device system includes a flexible tool having a first size and shape and a case for housing the flexible tool. When placed within the case, the flexible tool assumes a second, low profile size and shape that permits delivery through a flexible and articulated lumen of an elongate instrument.

The delivery devices described herein can be used with a variety of surgical or non-surgical systems, including, for example, those described in U.S. patent application Ser. Nos. 11/946,779; 11/946,790; 11/946,799; 11/946,807; 11/946,812; and 11/946,818, which are incorporated herein by reference. In one aspect, the delivery device is adapted for travel through a channel or lumen of an elongate flexible body, such as, for example, a catheter, endoscope, and/or guide tube. Such elongate devices for working at a location spaced from a user are referred to herein as a guide tube.

While the discussion of devices, systems, and methods below may generally refer to “needles” for convenience, the methods and devices described herein can deliver a variety of flexible, bendable, deformable, stretchable, compressible, unfolding, unrolling, and/or elastic tools, devices, and/or implants through a guide tube channel. For example, in addition to needles (including suture needles and other types), the devices, systems, and methods discussed herein are also applicable to clips, mesh, suture or ligature material, colonic stents, space creating frameworks or structures, grafts, patches, clips, tacks, biologics, fasteners, injection needles, knives (including sharp or pointed knives), and other surgical or medical devices to be used within a patient. The devices delivered by these methods and systems can be permanent, temporary, or absorbable. Further, the systems, devices, and methods apply to a wide range of practice areas, including gynecology, urology, palliative needs, and others.

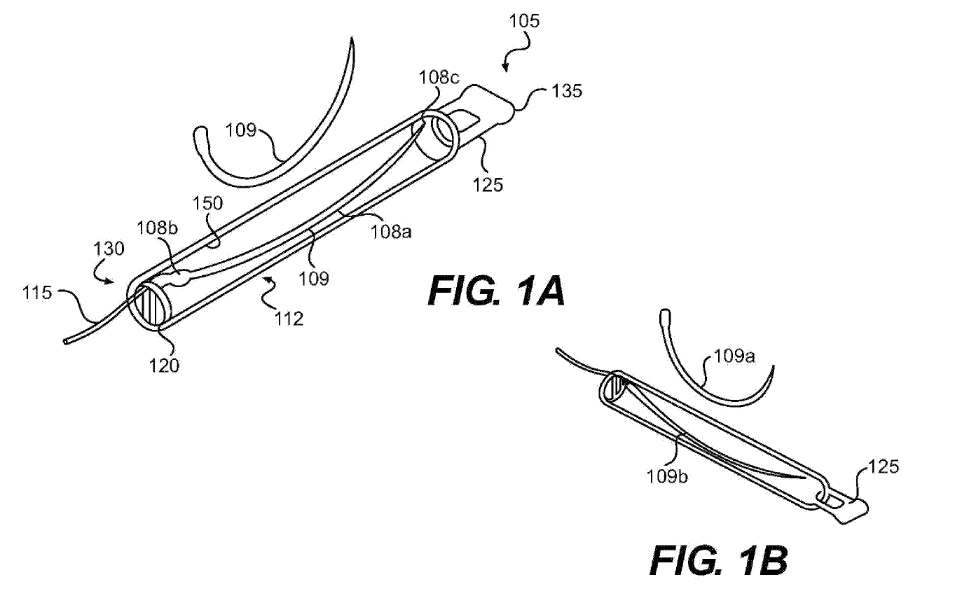

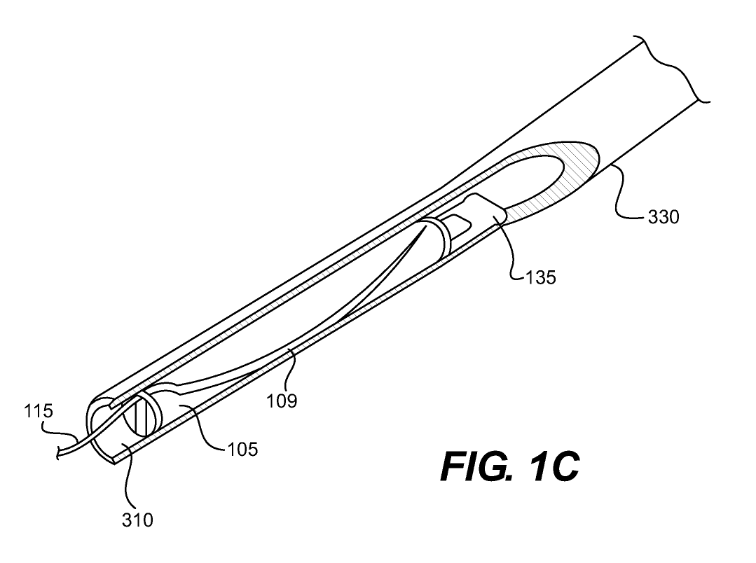

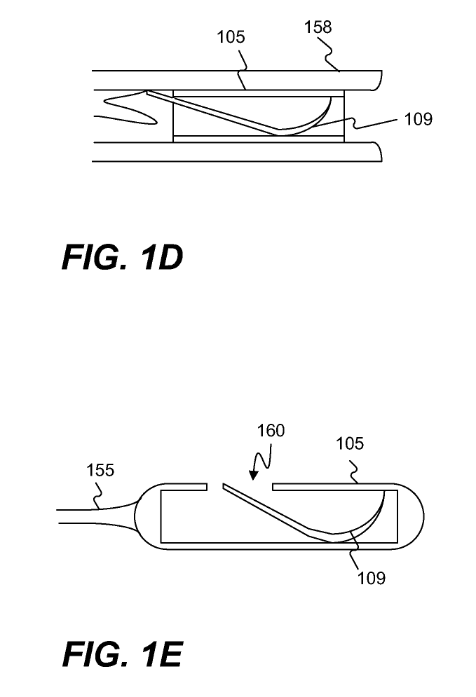

FIGS. 1A and 1B illustrate an exemplary embodiment of a case 105 for housing a flexible tool such as needle 109. FIG. 1C illustrates case 105 positioned within the lumen 310 of a guide tube 330. As shown in FIG. 1A, the needle 109 may have a first or operational shape and a second more compact shape when positioned within case 105. Case 105 can allow delivery of the needle through a lumen that is smaller than the width of the needle in the operational configuration. In addition, case 105 can reduce the chance of the needle snagging the inner wall of a lumen and becoming lodged therein, protecting both the tool and the lumen.

Case 105 can comprise an elongate body 112 with a first end 135 and a second end 130. In one aspect, first end 135 is a proximal end and second end 130 is a distal end. Body 112 can have an outer shape that corresponds in size and shape to the inner surface of a lumen extending through an elongate flexible instrument, such as a guide tube. The outer surface of case 105, in one aspect, can have a generally cylindrical shape. Alternatively, case 105 can correspond in size, but not shape, to the interior of a guide tube lumen. For example, the outer surface of the case can have a non-cylindrical shape, such as a polygonal cross-sectional shape or an irregular cross-sectional shape. It can accommodate a particular tool by having more space at one portion than at another portion of the container. Indeed, the body need not have a constant cross-sectional dimension. For example, body 112 can taper, be pill shaped, or otherwise have a variable diameter (i.e., width).

In one embodiment, the outer surface of body 112 can include alignment features (not illustrated) that inhibit relative rotational movement between case 105 and guide tube 330. For example, a longitudinal ridge or recess can extend along an outer portion of body 112 and ride along or in a recess or protrusion(s) that extends along all or a part of the length of the guide tube lumen. As case 105 moves through the lumen, surface features on the outer surface of body 112 and on an inner surface of the guide tube can control the orientation of case 105 with respect to the guide tube. While a recess and corresponding protrusion are disclosed, a variety of alternative surface features on the outer surface of case 105 and the inner surface of a channel could similarly inhibit rotation of the case within the channel. In another aspect, the shape of body 112 and/or guide tube lumen inhibit relative rotation. For example, body 112 can have an oval cross-sectional shape that corresponds to an oval shaped guide tube lumen. In one aspect, orientation can be identified by markings, either internal or external. The markings can be imageable with x-ray, MRI, ultrasound, or other methods in one embodiment.

Body 112 can be comprised of a variety of surgical and medical grade materials. In one aspect, at least a portion of case 105 is transparent or translucent to permit visualization of a tool within the case. The body 112 can have color markings that identify the tool location in another embodiment. Body 112 can also, or alternatively, be formed of a flexible or bendable material. In some cases, the guide tube is inserted through a tortuous pathway. The flexibility of body 112 can permit the case to bend around tight corners within the guide tube lumen. Alternatively, body 112 could be formed of a rigid or semi-rigid material where the length and/or width of the case allows the case to travel around bends within a lumen and/or in applications where a guide tube does not extends through tight corners. In one embodiment, a mix of materials comprise body 112. For example, one half could be metal while another half is glass or plastic.

The outer surface of body 112 can further include a lubricious material or coating to facilitate movement through a guide tube channel. For example, a fluoropolymer can define a portion of the body or be position over a portion of the body. Body 112 can additionally or alternatively include a variety of other lubricous or non-lubricous materials, such as, for example, silicon, polycarbonate or other type polymer, a composite, stainless steel or other type metal, titanium, Nitinol, a combination of parts and materials (e.g., a Nitinol tube/container with polycarbonate end caps), or an absorbable polymer (e.g., all or part of the body 112 could be left in place within the patient). The body can comprise additional materials as well, including PET, nylon, PVC, HDPC, and glass.

The proximal end 135 of case 105 can include a mating surface 125 to facilitate moving the case through a lumen and/or for controlling movement of the case upon exit from the distal end of a guide tube. As illustrated in FIGS. 1A and 1B, mating surface 125, in one exemplary embodiment, includes a “U” shaped member (e.g., handle or tab) extending from the proximal surface of body 112. The mating surface can act as a bumper against which a push rod rests to drive the case through a lumen. Alternatively, a push rod can mate with the case to allow pushing and pulling. Additionally, the mating feature 125 can facilitate handling of case 105 upon reaching the distal end of the lumen and/or after exiting the distal end of the guide tube. An articulating instrument can grasp mating surface 125 to hold case 105 in place and/or to move the case into a desired orientation and location. For example, a grasper could pinch surface 125 and mate there with. While a “U” shaped handle is illustrated, mating surface could alternatively include, for example, a tab, threaded connection, and/or hook. Alternatively or in addition, the case 105 could also have a tether or pull cable attached for retracting the catheter back into the working channel. The cable could be permanently or temporarily connected to the container. In one embodiment the mating feature includes a lock to the tether, and in another embodiment the tether can rotate when affixed to the container. In still another embodiment, case 105 does not include a mating surface at end 135. A push rod, instead, could contact a flat proximal end of the device to drive it through a lumen. Thus, case 105 can be devoid of a mating surface or the mating surface could be positioned at another location (e.g., the distal end of the case). In still another embodiment, case 105 could include multiple mating surfaces.

In another aspect, case 105 is delivered in the reverse orientation. End 135 can be delivered first and a push rod can contact end 130 to drive case 105 through a guide tube lumen.

Case 105 can also include at least one opening 120 for the passage of the needle into the case. In one aspect, opening 120 is positioned at the distal end of the case. Upon exiting a distal end of the guide tube lumen, needle 109 can be removed by pulling the needle out of opening 120. The opening can comprise different shapes or sizes sufficient for removing the needle from the case.

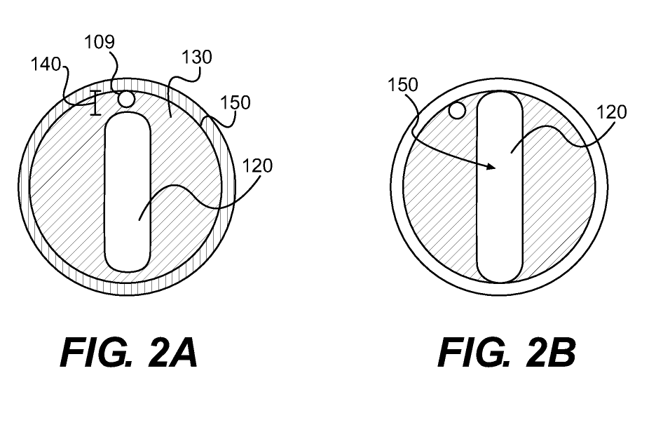

Referring now to FIGS. 2A and 2B, the opening 120 can comprise a slot. In FIG. 2A the slot does not extend the full width of the inner cavity. The size of the slot can prevent the needle 109 from slipping out of the case by providing a stop section 140 at the base of the opening. Alternatively, as illustrated in FIG. 2B, the slot can extend the full width of the case.

In another embodiment, the distal end 130 of case 105 is open and corresponds to the cross-sectional shape and size of inner chamber 150. In such an embodiment, the spring effect (discussed below) can hold the needle in place within the case. Alternatively, the needle can be tethered or mated with case 105 to inhibit unwanted egress of the needle. In one aspect, the needle can sit in a recess within the case. The recess can inhibit movement between the needle and case. In another aspect, the needle can be held with a mechanical connection, such as, for example, a snap fit between two surfaces of the inner chamber 150. Other exemplary connections include a latch, interference fit, and/or frictional engagement. In one embodiment, the inner chamber will comprise material softer then the needle tip to prevent damaging the needle tip. In another embodiment, the needle tip will be positioned away from the interior surface. For example, shoulders can house the needle tip, or the needle tip can pass through a slot in the container. In yet another aspect, the sharp or working end 108c of needle 109 can stick into and mate with a portion of case 105. For example, a soft polymeric substance can be positioned in the proximal end of inner chamber 150. The needle can be forced into and retained by the substance. Exemplary substances include a dense durometer elastomer (such as Santoprene), silicone rubber, a soft durometer polymer (such as Teflon), polyurethane, a dense durometer foam (such as silicone), foam, rubber, Poron, or even an absorbable polymer.

Case 105 can include an inner chamber 150 for housing at least a portion of needle 109. While the illustrated embodiment has a substantially cylindrical inner chamber, the shape of the inner chamber need not correspond to the shape of the outer surface of case 105. For example, the interior chamber can be contoured or shaped to maintain a particular orientation of the needle. In one aspect, chamber 150 has an elongate cross sectional shape that generally corresponds to the needle. For example, chamber 150 can have a shape similar to the slot opening illustrated in FIG. 2B. Thus, the chamber can prevent rotation of the needle within the chamber and afford greater predictability when removing the needle 109 from the case 105.

In one embodiment, bending or flexing the needle 109 into position for storage in the case 105 can create a spring effect that helps retain the needle within the case and/or inhibits relative movement of the needle with respect to the case. For example, referring now to FIG. 1A, various points 108a, 108b, and 108c may exert pressure upon the interior of chamber 150. The location of contact points 108a, 108b, and 108c can vary depending on the shape of the needle 109 and the degree to which it is bent/compressed. In the illustrated embodiment, needle 109 has contact point 108b at a first end, contact point 108c at a sharp second end, and contact point 108a at the apex of the bend in the needle. At point 108a, the needle exerts pressure on the opposite side of the inner cavity as compared to the first and second ends of the needle at points 108b and 108c. The forces applied on opposing surfaces of the inner chamber can cause needle 109 to remain substantially stationary in the case 105 during delivery. In general, the flexible tool can be held in such a position as to spring out or orient itself upon deployment from the case. For example, a fastener can spring out of the case and pierce tissue as a result of spring force in one embodiment.

In another embodiment, a cap (not pictured) is provided for covering the sharp end of the needle. The cap can receive at least a portion of the needle 105, preventing the needle 105 from falling out of the case. The cap can also provide a protective covering that prevents damage or wear to the needle 109 and the case 105.

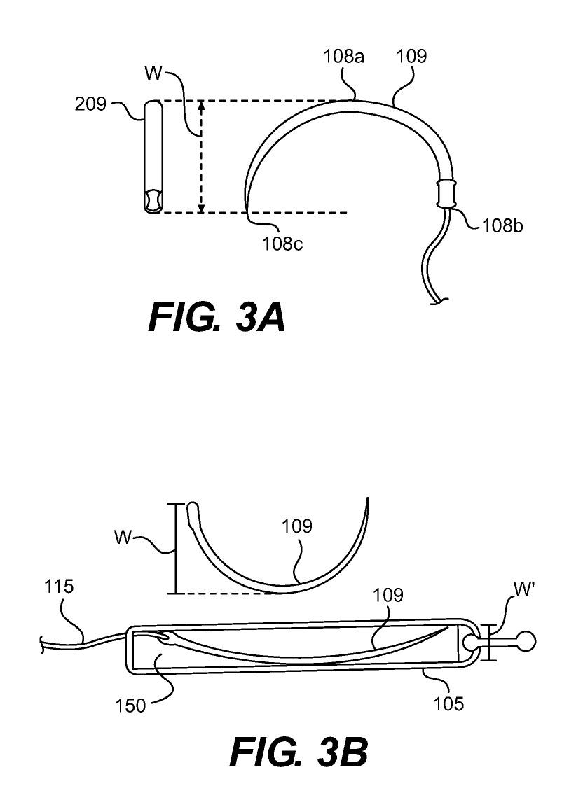

The needle 109 can have various shapes, such as the curved shape illustrated in FIG. 1A. Generally, needle 109 has a curved body. In particular, the needle can have a curvature over all or a portion of the needle body. In one aspect the needle includes a curved middle portion or tip end such as in a ski needle. The needle (i.e., flexible tool) can also have 3-dimensional curves, compound angles, and/or coils. In addition, the needle can have a constant radius of curvature or a non-uniform curvature that increases towards the sharp end of the needle. Needle shapes can include a 180 degree diameter curve (e.g., FIGS. 3A and 3B), a 260 degree curve or other shapes useful in performing surgical procedures. Depending on the type of procedure contemplated, one skilled in the art will appreciate that a variety of needles shapes and sizes can be used.

Needle 109 can be fabricated from a variety of surgical or medical grade materials. In one aspect, the materials used to form needle 109 allow sufficient flexing, bending, and/or deforming to assume a low profile shape when positioned within case 105. In addition, the material properties can permit the needle to return to its original shape after removal from the case. Alternatively, the needle can be permanently deformed when placed in case 105. If desired, the needle can be bent back to its original shape after removal from the case. This allows the transport of a flexible tool of any geometry, shape, material, or hollowness by transporting the tool as flat wire or in a “D” shape, for example.

In one embodiment, the needle 109 is created from an annealed or one-fourth hardened stainless steel. This composition allows a user to reset the needle 109 after removing it from the case 105 if the needle has been deformed. The user can reshape a single needle for multiple uses within a procedure and thereby accommodate the need for a particular bend that may arise during the course of a procedure.

One skilled in the art will appreciate that the needle can be constructed of various other materials depending on the desired rigidity, strength, length, and/or thickness. One skilled in the art will also appreciate that permanent or temporary coatings, shealths, covers, etc. could be applied to the flexible tool.

FIGS. 3A and 3B further illustrate how a needle can have a smaller spatial width when contained in the case 105. FIG. 3A includes a side view and a front view of needle 109. The spatial width (or radius height) W of the needle is measured from a base line between end 108c and opposite end 108b of the needle 109 to a parallel line that intersects the top most point 108a of needle 109. In this way, the spatial width W (i.e., radius height) represents the maximum opening width required to accommodate the needle 109 within a lumen when the needle is oriented to minimize the maximum width.

Turning to FIG. 3B, the difference between the width of needle 109 outside of case 105 and contained within case 105 is shown. In this example, the case 105 has an inner cavity with a width that is smaller than the spatial width W of the needle 109. However, as illustrated, the needle 109 can fit inside the case 105 when flexed or bent into the second shape with a smaller spatial width W′. In this example, the smaller spatial width W′ is substantially equivalent to the width of the inner cavity 150.

In one embodiment, it is possible to pass a curved suturing needle through a catheter having a diameter smaller than the spatial width of the needle in an unconstrained configuration. (The use of the term “diameter” does not imply that the catheter opening must be round. Other opening shapes are also contemplated, and the term “diameter” should be understood to refer to a width of the opening.) In one aspect, the spatial width of the needle is greater than 1.3 times the minimum diameter of the catheter, in another aspect, greater than about 1.5 times the minimum diameter, and in still another aspect, greater than about 2-3 times the minimum diameter. For example, a needle with a spatial width of approximately 10 mm can be delivered through a catheter with a diameter of 4 mm by enclosing the needle within the case 105.

The length of the inner cavity of case 105 can be sized for receipt of needle 109. In one embodiment, the entire needle 109 is contained within the inner cavity of the case 105. Alternatively, the end 130 of the needle (e.g., a blunt end) can extend outside of case 105.

FIG. 1C, as mentioned above, is an exemplary cross-sectional illustration of lumen 310 containing case 105. Case 105 houses a needle in the second, lower profile shape, and the case is small enough to slide through the passageway of lumen 310.

The case 105 can be pushed down the lumen 310 with a push rod or push tube. The push rod can be attached to the case 105 in one embodiment. For example, FIG. 1E illustrates one such implementation, with push rod 155 affixed to the case 105. In another aspect, a cord or cable is attached to the case 105 to facilitate retrieving and/or withdrawing the case 105 from the target area. But some implementations do not require retrieving the case, such as when the case is biodegradable. In still another aspect, the case and/or needle are stored in or at the distal end of the guide tube after use and are removed with the guide tube. In yet another aspect, the needle is re-positioned within the case after use and removed with the case. The case can be removed via mechanical, fluid, electrical, or heat-based means, any of which can act as a triggering mechanism for removal.

The tool 109 can also extend partially out of the case 105, such as shown in FIGS. 1D and 1E. This can accommodate a tool 109 with a larger cross sectional width than the inner cavity of the case. In one aspect, this can allow transporting the tool without reshaping it to fit within the container (i.e., case). For example, the tool 109 depicted in exemplary FIG. 1D extends out the proximal end of the case 105. This allows for transporting the tool 109 in case 105 down lumen 158 without risking the tool snagging on lumen 158. Alternatively, exemplary FIG. 1E illustrates a case with a window 160 in the wall of the case through which a portion of the tool 109 extends.

Turning back to FIG. 1C, in one embodiment, suture material 115 can be supplied with needle 109. For example, suture material 115 can be pre-attached to needle 109 and positioned within case 105. In one aspect, the suture material can also extend at least partially out of inner chamber 150 such that a tool can grasp the suture material and remove the needle by pulling on the suture material. In another embodiment, where case 105 is delivered with end 135 positioned distally, suture material can extend through the lumen from the needle to a proximal portion of the guide tube. As the case is moved through the lumen, suture material can be fed through the guide tube lumen. When the suturing is complete, the user can sever the suture material near the distal end of the guide tube lumen and pull the excess suture material back through the lumen, removing it from the patient's body.

While the above embodiment of case 105 includes an aperture for insertion/removal of the needle, other configurations are contemplated. For example, case 105 can include a “door” or removable panel.

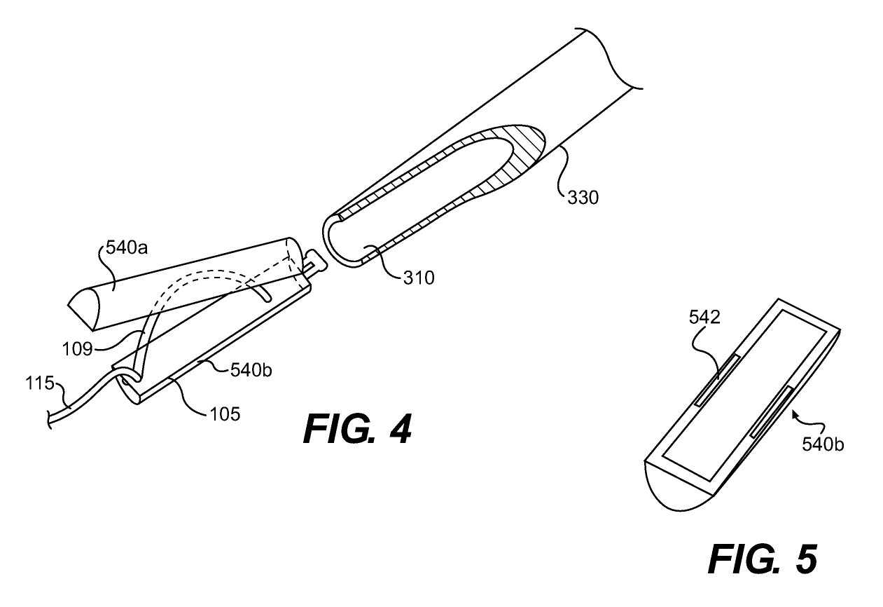

Turning to FIGS. 4 and 5, another embodiment of case 105 is disclosed. Case 105 comprises a first section 540a and a second section 540b that can open or separate to allow delivery of the needle. In one aspect, the first and second section are detachably mated with one another. In another aspect, the first and second sections are movably mated with one another. The sections can be mated with a tabbed latch, spring loaded latch, magnetic key and slot, and other means. In one embodiment, one section of the case (such as the door) could be removed while the base portion of the case stays in place. The removed portion could be left to dissolve within the patient.

In one embodiment, the case of FIGS. 4 and 5 is self-opening. When positioned within the lumen 310 guide tube 330 can constrain the first and second sections of case 105 and prevent opening of the case. Once the case is delivered through the lumen and is no longer constrained, the first and sections can move relative to one another to open case 105 and deliver the needle. In one embodiment, the two sections 540a and 540b are forced apart by the spring effect of the needle 109.

With respect to FIG. 4, case 105 can includes a hinge between the two sections 540a and 540b. Upon exiting lumen 310 the first and second sections can pivot away from one another to open case 105. This feature can facilitate reloading the case 105 into the lumen 310 for retrieval without having to realign the case sections 540a and 540b.

In another embodiment, the case sections 540a and 540b completely detach from one another. The case section can mate with each other in a manner that inhibits relative movement of the case sections while positioned within lumen 310. For example, as illustrated in FIG. 5, a recess 542 on the surface of section 540b can mate with a protrusion on section 540a (not illustrated). The mating features can inhibit relative longitudinal and/or rotational movement of sections 540a, 540b. However, the mating features can allow radial movement of sections 540a, 540b away from one another. While positioned within the lumen, the walls of the lumen can inhibit radial movement of sections 540a, 540b relative to one another and prevent case 105 from opening. Thus, the lumen 310 can constrain case 105 and inhibit opening of the case as it travels through the guide tube. Once the case exits the lumen, the walls of the lumen no longer constrain the case and the case can self-open.

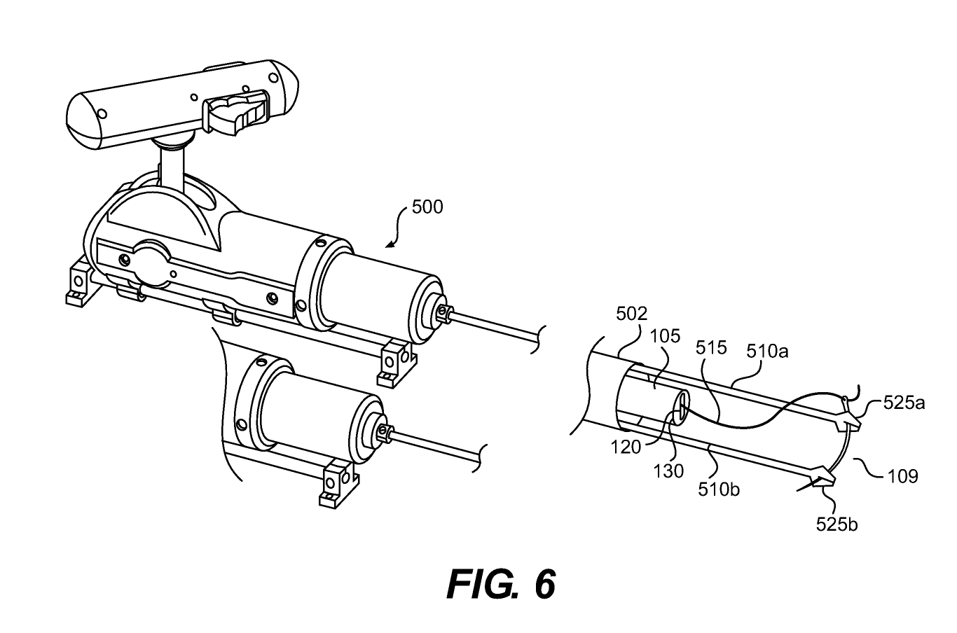

FIG. 6 is an exemplary illustration of a system 500 for use with needle 109. In the illustrated embodiment, system 500 allows control of two, three, four, or more than four degrees of freedom of the distal end of instruments 510a and 510b for performing actions at a procedure site within a patient. System 500 can further include a guide tube 502 having at least one lumen. In one aspect, each of instruments 510a, 510b, and case 105 can pass through a lumen in guide tube 502. The instruments can pass through the same or different lumens. While two instruments are pictured, system 500 can include a single instrument or more than two instruments. For example, Boston Scientific's DDES System could be used to deliver the case, remove and/or manipulate the flexible tool, and/or return the case after a successful procedure.

The distal end of each instrument 510a and 510b can comprise end effectors such as graspers 525a and 525b. The graspers can be used to position case 105, to remove the needle from the case, and/or to bend the needle 505 into a particular shape.

In one embodiment, needle 109 is pulled out of the case by grasping and pulling the suture material and/or an end of the needle. In this embodiment, the case 105 can require an opposing force to hold the case in place. For example, one grasper 525a can squeeze the handling feature of the case, while the other grasper 525b pulls the needle out.

In another embodiment, the push rod or a cable remains attached to the case and a single grasper can remove needle 109 while the push rod provides a counter force. For example, in FIG. 6, the case 105 protrudes from guide tube 330 with the open end 130 (and opening 120) exposed. In this embodiment, the user can remove the needle 109 by directly grasping the needle or by pulling on the suture material 515.

However, in another embodiment, the case has an opposite orientation, wherein the handling feature is positioned distally. In such an arrangement, one of the instruments can be used to first engage the handling feature on the case and to pull case 105 out of lumen 330. After the case 105 is removed, the needle is removed from the case 105.

In one embodiment, grasping effectors 525a-b could apply a crimp that is transported in the case to the procedure site. Surgical clip devices or parts, which could be transported to the procedure cite in multiple parts, could be placed in the container in an “open” or straight condition, deployed out (e.g., pulled, ejected, etc), grasped, and placed by a DDES or other similar device(s). The DDES or other system could be used to return the tool(s) into the case(s) by manipulating the case(s) and/or tool(s) (e.g., by closing, squeezing, clipping in place, locking, spring closing, ratcheting, manipulating a threaded-type connection, etc.).

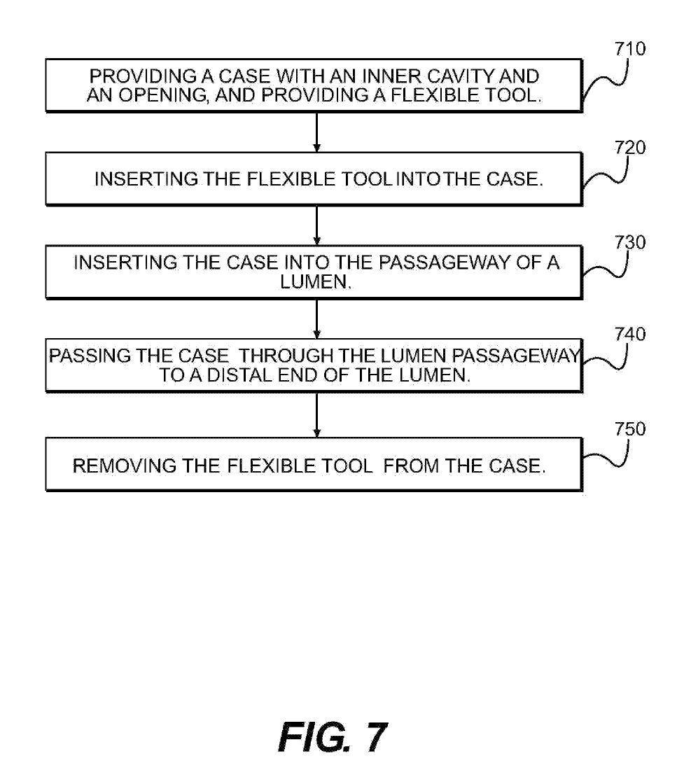

FIG. 7 is an exemplary flow chart including steps taken for delivering a needle to a procedure location. These steps and others are also apparent from the above disclosure, but are presented here for clarity.

Step 710 includes providing a case with an inner cavity and an opening. In one embodiment, the opening is positioned at one end of the case and a handling or mating feature is positioned at the other end of the case.

At step 720, a needle is inserted into the case through the opening. In one embodiment, this step is accomplished during manufacturing, such that each case contains a needle upon delivery to the customer. This approach maintain needle sterility until the case is opened or until the case is removed from a sterile package. However, in another embodiment, a user (e.g., doctor, surgeon, or nurse) inserts the needle into the case in preparation for a particular procedure. In yet another embodiment, the user inserts multiple needles into the case. Other items, such as suture material can also be inserted into the case.

At step 730, the case is inserted into the passageway of a lumen, such as a catheter or guide tube. The insertion typically occurs at the proximal end of the lumen, outside of the patient's body. Alternatively, the case could be placed in the body through a PEG or port.

At step 740, the case is passed through the passageway of the lumen to the distal end of the lumen. The orientation of the case inside the lumen can vary in an embodiment. The user can use a push rod or cable to push or pull the case to the distal end of the lumen.

At step 750, the needle is removed from the case. In one embodiment, a tool is used to pull the needle out of the case. For example, the tool can include a grasper that grabs and pulls either the needle or a suture material that is attached to the needle.

Further steps are also possible. For example, the user can bend the needle after removing it in one embodiment.

Additionally, the step of returning the case and/or needle to the proximal end of the lumen can also be an included step in one embodiment. This can include inserting the needle into the case and pulling the case proximally through the lumen.

Other embodiments will be apparent to those skilled in the art from consideration of the specification and practice of the embodiments disclosed herein. It is intended that the specification and examples be considered as exemplary only, with a true scope and spirit of the embodiments being indicated by the following claims.

A variety of control members, which allow a distal end of tool 40 to be actuated in the up/down, right/left, forward/backward, and rotational directions, can be used to manipulate the needle and/or case at the procedure location. Some exemplary control mechanisms are disclosed, for example, in U.S. patent application Ser. No. 11/165,593, entitled “Medical Device Control System” and U.S. patent application Ser. No. 11/474,114, entitled “Medical Device Control System,” both of which are hereby incorporated by reference in their entirety.

1. A method for delivering a flexible tool to the site of a procedure, the method comprising:
inserting a case into a lumen of an elongate flexible device, the lumen having a proximal end and a distal end and a minimum interior width, the case having an inner cavity and a flexible tool positioned within the cavity, the tool having a first shape with a first spatial width while inside the case, the first shape different from a second shape when the flexible tool is unconstrained by the case;
passing the case through the passageway to the distal end of the lumen; and
removing the flexible tool from the case and allowing the tool to assume the second shape, the second shape having a spatial width greater than the minimum interior width of the lumen.

2. The method of claim 1, wherein the flexible tool is a suture needle.

3. The method of claim 2, wherein the second shape is the needle's natural shape prior to insertion into the case.

4. The method of claim 1, further including the step, subsequent to removing the flexible tool, of bending the flexible tool.

5. The method of claim 1, wherein removing the flexibile tool is performed by pulling a suture material attached to the flexible tool.

6. The method of claim 1, wherein the case is self-opening, and the step of removing the flexible tool includes moving the case out of the distal end of the lumen and allowing the case to open.

7. The method of claim 1, wherein the elongate flexible device is a guide tube comprising a lumen and at least one articulating instrument comprising a proximal controller, an elongate flexible body, and a distal grasper controlled by the proximal controller.

8. The method of claim 7, further comprising, after the step of passing, the step of grabbing the case with the distal grasper of the articulating instrument.

9. The method of claim 8, wherein the step of removing the tool includes grabbing the tool with a second articulating instrument.

10. A system for delivering a tool through a lumen of an elongate device, comprising:
an elongate device having at least one lumen;
a case comprising an outer surface, a proximal end, a distal end, and an inner cavity, the outer surface sized to fit within the at least one lumen; and
a tool comprising a proximal end and a sharp distal end, the tool having a curved shape and a spatial width larger than a cross sectional width of the cavity, the tool being situated inside the case in a compressed manner.

11. The system of claim 10, wherein the case comprises a first section and a second section and is self opening when removed from the at least one lumen.

12. The system of claim 10, wherein the case comprises a first section and a second section that are detachably mated.

13. The system of claim 10, wherein the case comprises a first section and a second section that are mated with a hinge.

14. The system of claim 10, wherein the proximal end of the case includes a handling feature.

15. The system of claim 10, wherein the distal end of the case is open.

16. A case for delivering a flexible tool through a lumen comprising:
an outer surface; and
an inner chamber for holding the flexible tool, wherein the inner chamber has a cross-sectional width that accommodates the tool, wherein the cross-sectional width of the inner chamber is less than a cross-sectional width of the tool after the tool is removed from the inner chamber.

17. The case of claim 16, wherein the outer surface is comprised of two sections coupled to one another, and decoupling the two sections releases the flexible tool from the inner chamber.

18. The case of claim 16, wherein the outer surface includes a window through which a portion of the tool extends out of the inner chamber.

19. A tool for delivering in a case to a procedure site within a patient, the tool comprising:
a first cross-sectional width when inside the case; and
a second cross-sectional width when removed from the case, the second cross-sectional width being greater than the first cross-sectional width.

20. The tool of claim 19, wherein the tool further comprises a curved shape that bends when placed inside the case.

21. The tool of 19, wherein the tool is a needle.
